(19) United States
(12) Patent Application Publication
(10) Pub. No.: US 2023/0134699 A1
(43) Pub. Date: May 4, 2023

(54) HYBRID MICRONEEDLE ARRAYS

(71) Applicant: Carnegie Mellon University, Pittsburgh, PA (US)

(72) Inventors: O. Burak Ozdoganlar, Pittsburgh, PA (US); Ant Yucesoy, Pittsburgh, PA (US)

(73) Assignee: Carnegie Mellon University, Pittsburgh, PA (US)

(21) Appl. No.: 17/917,808

(22) PCT Filed: Apr. 9, 2021
(86) PCT No.: PCT/US2021/026722

§ 371 Date: Oct. 7, 2022

Related U.S. Application Data
(60) Provisional application No. 63/007,473, filed on Apr. 9, 2020.
(60) Provisional application No. 63/080,208, filed on Sep. 18, 2020.

(51) Int. Cl. A61B 5/15

(52) U.S. Cl. CPC A61B 5/150015; A61B 5/150984; A61B 5/150282

(57) ABSTRACT

A hybrid microneedle array and a method of fabricating the array is used for delivery of drugs, vaccines, and other therapeutic agents into tissues, including skin, heart, inner ear, and other tissues. The microneedle array can facilitate precise and reproducible intradermal delivery. Each microneedle has a dissolvable tip with a hollow body permitting the delivery of a variety of therapeutic agents into the skin. A fabrication process utilizes a two part mold to separately mold a dissolvable tip and a solid body portion of each microneedle in the array.

### CROSS-REFERENCE TO RELATED APPLICATIONS

This application claims the benefit under 35 U.S.C. § 119 of Provisional Application Ser. No. 63/007,473 filed Apr. 9, 2020, and Provisional Application Ser. No. 63/080,208 filed Sep. 18, 2020, each of which is incorporated herein by reference.

### STATEMENT REGARDING FEDERALLY SPONSORED RESEARCH

Not applicable.

### BACKGROUND OF THE INVENTION

The present disclosure is related generally to drug delivery and fluid sampling systems. More specifically, the disclosure is related to microneedle arrays used to deliver drugs, vaccines, therapeutics, and other bioactive and bio-reactive compounds both to the skin (i.e., intradermally) and to other tissues in a precise and controllable manner.

Hypodermic needles have long been used for delivering therapeutics into and sampling fluid from the human body. The drawbacks of hypodermic needles include pain at the injection site, potential tissue damage associated with needle insertion, the possibility of transmission of infectious diseases through needle reuse, and accidental needlestick injuries to health-care professionals. While intradermal delivery is an ideal route, the traditional Mantoux intradermal delivery technique using hypodermic needles requires training and skill to perform and can be unreliable and inconsistent for delivering desired quantities of antigen to the skin. Transdermal drug delivery is an alternative method for achieving systemic or localized pharmacological effects that eliminates the risk of needle injuries. The main challenge associated with this approach is sufficient drug delivery across the skin at therapeutically significant rates due to the barrier posed by the skin and its uppermost layer, the stratum corneum.

More recently, microneedle arrays (MNAs) have been demonstrated to transdermally deliver a broad range of drugs, biologics, and vaccines and offer advantages over hypodermic needles or transdermal patches. The high density of dendritic cells present in skin directly connect to the lymphatic system and activate the body's immune system to a higher degree than traditional intra-muscular injections. An MNA uses microscopic needles that create transport pathways by penetrating through the stratum corneum into the viable epidermis of the skin, short of the dermis layer with its nerves and vasculature. Hence, minimally invasive, bloodless, and painless application is possible with minimal tissue damage while enabling controlled delivery over time. When desired for specific applications, MNAs with longer needles can be used to reach vasculature or nerves. MNAs can enable a more efficient, highly reproducible and reliable route for clinical intradermal applications.

However, current MNA technologies have several limitations that preclude their use as effective drug delivery vehicles. For dissolvable MNAs, the volume of drug delivered to the skin is limited (commonly less than 1 μl per array), delivery rates are inconsistent, and only dryable therapeutics can be use. For instance, live cells (e.g., stem cells) cannot be delivered using dissolvable MNAs. Moreover, interaction of the drugs with dissolvable materials can prevent the desired biological effect. Encapsulation of the vaccine within the dissolvable material necessities γ-irradiation for sterilization, leading to a significant decrease bioactivity on a range of proteins, drugs, and viral vectors.

Similar to MNAs, arrays of hollow microneedles can be used to deliver a larger drug volume intradermally, but hollow microneedles suffer from the clogging of bores upon skin entry, higher forces and tissue damage during insertion, and the lack of precise control for delivery depth and amount. Therefore, it would be advantageous to develop an intradermal delivery system that permits the precise delivery of therapeutic agents in liquid or solid form with reduced harm to the patient's body.

### BRIEF SUMMARY

According to embodiments of the present disclosure is a hybrid microneedle array that can allow for the injection of vaccines, drugs, proteins, live cells, particulates, and other bioactive agents into skin or other tissues such as mucosa membranes (buccal delivery), cardiac muscle tissue, and suprachoroidal space through ocular tissue of the eye in a precise and distributed manner. Each hybrid microneedle has a dissolvable tip with a hollow body. The hollow body, which is also referred to as a micro-cannula, can be made from a non-dissolvable material, or a material that will dissolve in a significantly slower than that of the tip material. The dissolvable tip permits low force, easy, and minimally damaging penetration of each microneedle of the array through the outer layer of the skin to deliver the therapeutic agent to a targeted position of the tissue, e.g., the targeted layer of the skin. After penetration, the tip dissolves and a drug or other material can be delivered through the hollow body into the skin or other tissue.

In alternative embodiments, the hybrid microneedle array is attached to a standard syringe using an adaptor (which can be co-fabricated), giving a health care provider precise control over the amount of material injected into the patient. In yet another alternative embodiment, the hybrid microneedles are integrated into a blister-pack type of self-contained device with an embedded reservoir that includes the drug to be delivered. In these embodiments, in addition to a health-care provider, a patient can self-administer the hybrid MNAs to the skin. In another embodiment, a drug or a compound can be integrated into the dissolvable tips for delivery as a second-phase drug in addition to the bio-cargo delivered through the microcannulas. Hybrid MNAs can further be used for sampling liquids, such as blood or interstitial fluid, from the body for use in subsequent diagnosis purposes.

The hybrid MNA is especially effective in treatment of local skin ailments (e.g., dermatitis), skin cancer (e.g., melanoma, squamous cell carcinoma, basal cell carcinoma), and autoimmune conditions (e.g., psoriasis). Similarly, the hybrid MNA can be used for delivering Botox, Vitamin A, or similar chemicals/biologicals/compounds for cosmetic and other applications.

The disclosure is further directed to a method of fabricating the hybrid microneedles. The method uses a micromolding process, where the dissolvable tip is first molded then joined with a separately molded body portion. The master and production molds are created through a variety of techniques, including mechanical micromilling, diamond micromilling, micromolding, additive manufacturing, lithography, or a combination of such techniques. In addition, the fabrication method enables creation of adaptors, either fabricated separately or in conjunction/simultaneously with the hybrid MNAs, to enable attaching standard syringes or self-contained devices with the hybrid-MNAs.

### BRIEF DESCRIPTION OF THE SEVERAL VIEWS OF THE DRAWINGS

FIG. 1 shows a hybrid microneedle, according to one embodiment.

FIGS. 2A-2B show a microneedle array (FIG. 2A) and a detailed view of a microneedle in the array (FIG. 2B).

FIGS. 3A-3D show a microneedle array used with an adapter permitting use with a syringe (FIGS. 3A-3B), a dispenser (FIG. 3C), and a co-fabricated adapter (FIG. 3D).

FIGS. 4A-4C show various adapted used to connect the microneedle array to a syringe.

FIG. 5 is a flowchart identifying the steps of fabricating a microneedle array.

FIGS. 6A-6B are images of master molds used in the fabrication process.

FIG. 7 is a diagram of one step of the fabrication process.

FIG. 8 is an embodiment of the microneedle array used for drug delivery to the inner ear.

### DETAILED DESCRIPTION

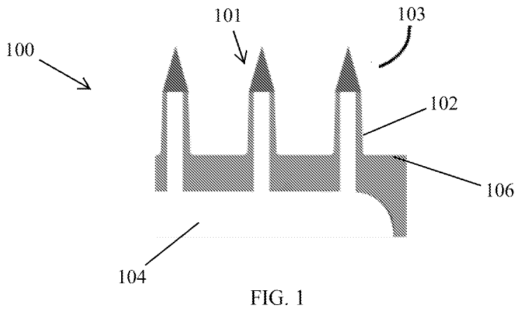

According to embodiments of the disclosure is a hybrid microneedle array 100 used for drug delivery and fluid sampling from a variety of tissues. Throughout the disclosure and in discussion of example embodiments, the skin is identified as the tissue of interest, but the microneedle array can be use on several tissue types. FIG. 1 is a cross-sectional view of an array 100 having a plurality of microneedles 101, where each microneedle 101 comprises a hollow body 102 and a dissolvable tip 103. Further shown is a reservoir 104 that may be used to store a drug, vaccine, or other therapeutic agent in a dried (or lyophilized form) or in a liquid form prior to use. Each microneedle 101 has a microbore traversing the longitudinal axis of the body 102, with an opening at a distal end adjacent to the tip 103 and a second opening at a proximate end in communication with the reservoir 104. The body 101 is fabricated from a solid, biocompatible, non-dissolvable material, such as a UV cured resin. In another embodiment, the body can be fabricated from a dissolvable material with a long dissolution profile, i.e., very slow dissolving.

When the microneedle 101 penetrates the skin of a patient, the tip 103 dissolves in a short period of time and the drug may flow from the reservoir 104 through the hollow body 102, exiting the distal end of the body 102 and into the skin of a patient. Prior to use, the solid tip 103 prevents the drug from being dispersed from the microneedle array 100. Additionally, a thin layer of poly(lactic-co-glycolic acid) (PLGA) or similar material can be included within the hollow body 102 at the distal end, behind the dissolvable tip 103 to prevent premature dissolution of the tip 103 before application due to the exposure to liquids in the reservoir.

In addition to drugs, the microneedle array 100 is capable of delivering many types of vaccines (including RNA, DNA, and protein-based vaccines, replication-competent vaccines, and live-attenuated vaccines), live cells (e.g., stem cells), viral vectors (e.g., for gene therapy), and peptide hormones (e.g., insulin) in a liquid form. The liquid to be delivered can be encapsulated in the integrated reservoir or remain in an external reservoir (e.g., a syringe or a blister pack) until delivery. The system also allows a solid-form drug loaded in the reservoir to be mixed in situ with a liquid phase (e.g., saline) during the delivery. For example, the hybrid microneedle array 100 allows a stable, lyophilized (dry) vaccine to be loaded into the integrated reservoir 104. In one embodiment, the lyophilized formulation is added to the reservoir 104 after slightly hydrating, compressing (or centrifuging) to fill the reservoir 104, and then drying while loaded in the array 100. Alternatively, the vaccine can be loaded into the reservoir 104 as a liquid formulation and then lyophilized in place. Similarly, other dry drug formulations can be incorporated into the reservoir 104.

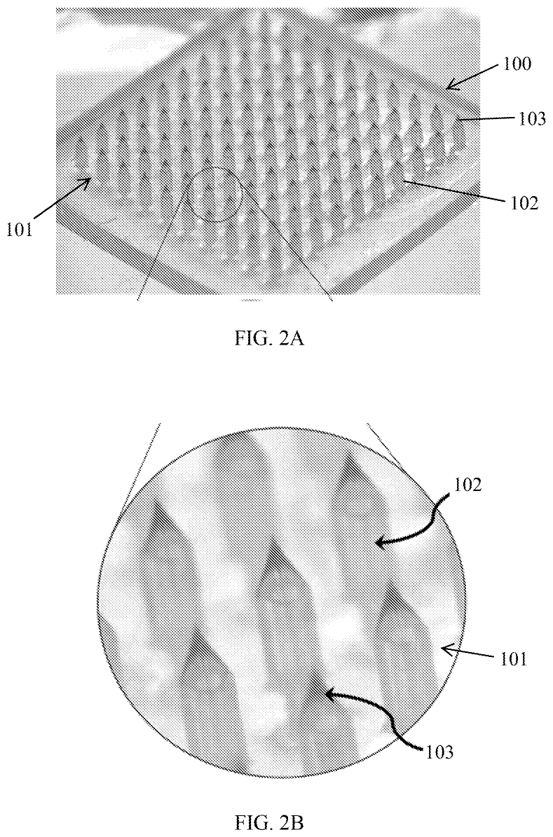

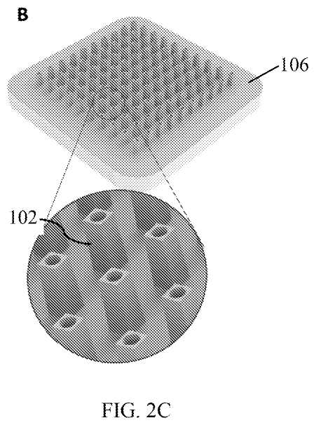

To attain low force, clog-free, and precise administration with minimal tissue damage, each microneedle 101 includes a sharp, dissolvable tip 103. As will be discussed in greater detail, the tip 103 is fabricated through a molding process that enables a purposeful design of the tip and precise control of the shape (e.g., including tip sharpness, apex angle, and cross-sectional geometry). In the embodiment shown in FIGS. 2A-2B, the tip 103 is a pyramid-shaped tip 103. In alternative embodiments, the tip 103 may have a cone, arrow, triangular, incurvate, or ovate-shaped tip 103. Further, the size may vary and in some embodiments the diameter or width of the tip 103 may be larger than the microcannula body 102 to create an undercut or a temporary retaining feature. By precisely controlling the tip shape, the microneedle array 100 is capable of penetrating the skin with little damage and permits a variety of materials to be used. In many prior microneedle arrays, the tips are limited to certain materials that are too fragile to consistently penetrate the skin. Unlike other tip fabrication methods (e.g., dip-coating), the molding-based tip fabrication methods of the present disclosure enable precise control of the tip shape to create effective, efficient, and failure free penetration of the hybrid microneedles 101 into the tissue.

The dissolvable tip 103 can be made from biocompatible and biodissolvable/biodegradable polymers, which dissolve or degrade after penetrating the skin. FIG. 2C shows the non-dissolvable body 102 of each microneedle 101 without the tip 103. The biocompatible polymer may include, for example, carboxymethylcellulose. Other biocompatible and biodissolvable/biodegradable materials can be used, including, for example, poly vinyl alcohol (PVA), simple sugars such as glucose or dextrose, hyoluruonic acid, trehalose, PLGA, and other similar materials, or the combination of two or more biocompatible materials. These materials offer strong adhesion to the body 102 of the microneedle 101 and are capable of forming sharp tips 103. In one embodiment, the tips 103 are capable of carrying encapsulated drug payload as a secondary set of drugs or vaccines to be delivered. While the sharp tips 103 penetrate the outer layer of the skin, the length of the microneedles 101 are short enough to prevent entering into the deeper, vascularized layers of the skin. As are result, the microneedle array 100 is painless and causes minimal trauma to the tissue. Due to the minimally invasive nature of the microneedle array 100, the array 100 can be used to deliver into delicate areas such as the eye by targeting the suprachoroidal space or cardiac tissue during surgery. Since the needle 101 length is customizable, in applications where deeper delivery, e.g., to the vasculature, nerves, or subcutaneous tissue, is desired, the needle body 102 can be lengthened to reach those tissue locations.

In the example embodiment shown in FIGS. 2A-2B, the array 100 is an 10×10 mm square with one-hundred needles 101 arranged in a 10×10 grid. Each needle 101 has a length of 1220 μm long and a width of 250 μm and a dissolvable tip height of approximately 500 μm. Variations in the geometry, cross section, height, and width may be made depending on the application, target delivery location, and depth, as well as the bio-cargo. For example, the angle of the microneedles 101 depicted in the embodiments of FIG. 1 and FIG. 2A are perpendicular; in alternative embodiments, the angle is non-orthogonal, e.g., including a negative bevel angle to retain the needles 101 in place when applied. Similarly, the cross-sectional shape can be square, circular, or any other shape. The microneedle array 100 may further have variations in the array 100 size, grid count, and spacing, and spatial arrangement. Indeed, a person having skill in the art will appreciate the need to adjust the height of the needles 101 to target specified delivery depths and thus, desired skin microenvironments.

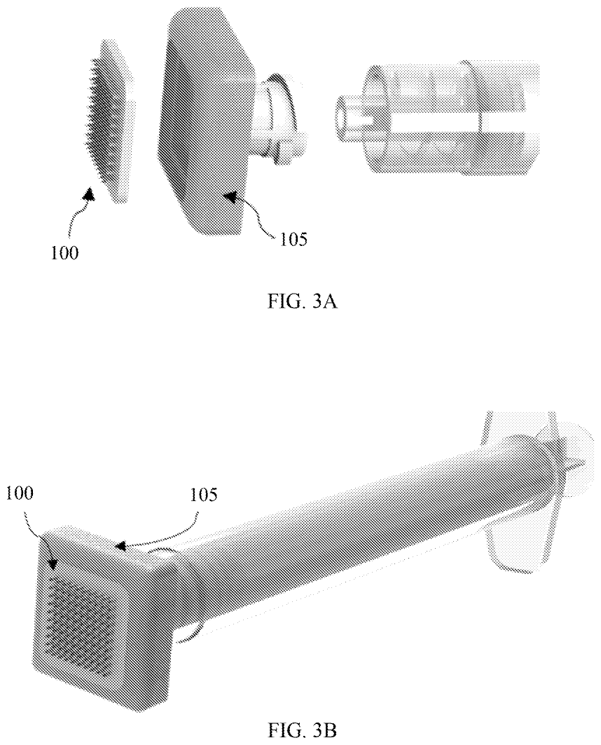

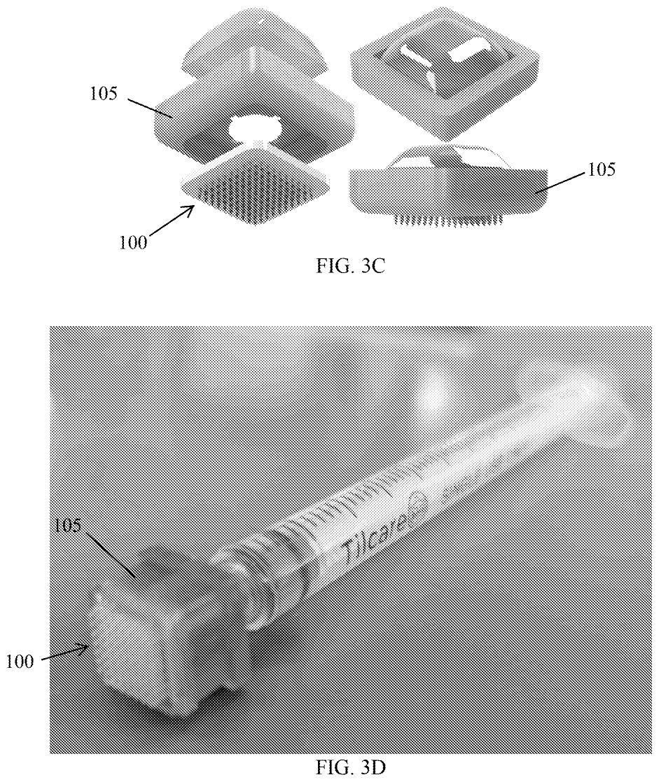

During use, the drug or vaccine stored in the reservoir 104 will diffuse through the hole in the body 102 into the skin after the tips 103 penetrate the skin and dissolve. However, in an alternative embodiment, the microneedle array 100 is fitted with an adapter 105 to allow the array 100 to be used with a standard syringe, as shown in FIGS. 3A-3B. The adapter 105 has a fitting (also referred to as an adapter) on one end that connects to a syringe. The other end of the adapter 105 has a recess in which an array 100 is placed. A fluid dispelled from the syringe flows through the adapter and into the reservoir 104 of the microneedle array 100. The fluid then flows through the body 102 of each microneedle 101 in the array 100, entering the patient's body. FIG. 3C shows an alternative use of the adapter 105, where the cargo and delivery method are integrated. By pushing on the backside of the adapter, the cargo is dispensed through the microneedle array 100 without the use of external equipment. FIG. 3D shows an adapter 105 that is co-fabricated with the array 100. In the example embodiment shown in FIG. 3D, the adapter 105 is molded separately, then placed into the body molds when the body 102 is created. Co-fabricating the adapter 105 aids accurate adhesion to the body 102.

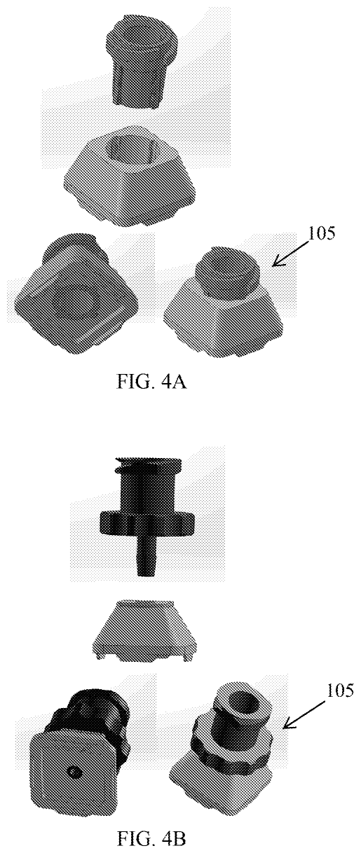

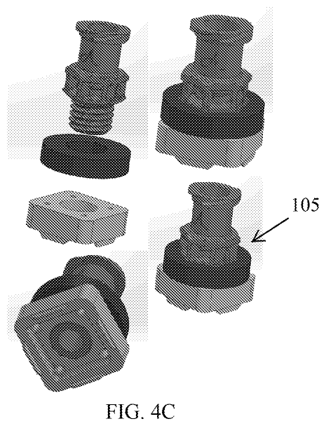

FIGS. 4A-4C shows variations of the adapter 105. With the use of a syringe, the amount of therapeutic agent delivered through the microneedle array 100 can be significantly higher (e.g., 100s of times) than typical fully-dissolvable microneedle arrays and the rate of administration can be precisely controlled. The adapter 105 can also be used to connect the array 100 to a 3-way stopcock and subsequently a syringe through Luer connections. Thus, the delivery of drugs can be performed by way of passive diffusion (e.g., time release) or instantaneous injection. Similarly, a blister pack or other self-contained delivery device can be integrated with hybrid microneedles as a drug delivery system.

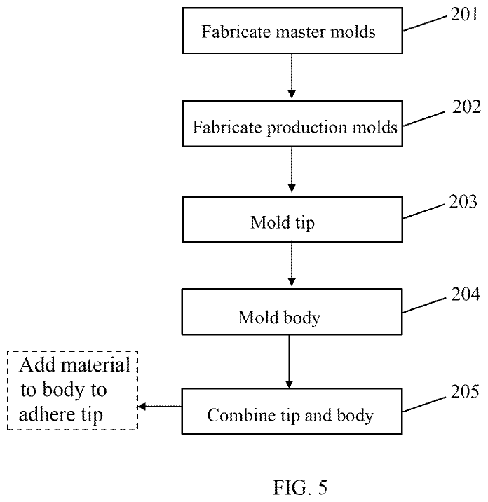

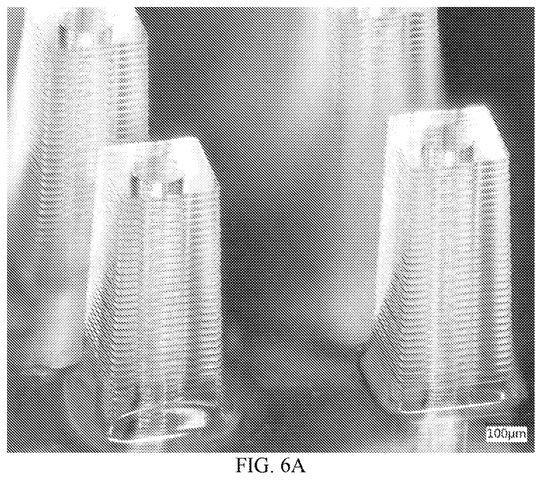

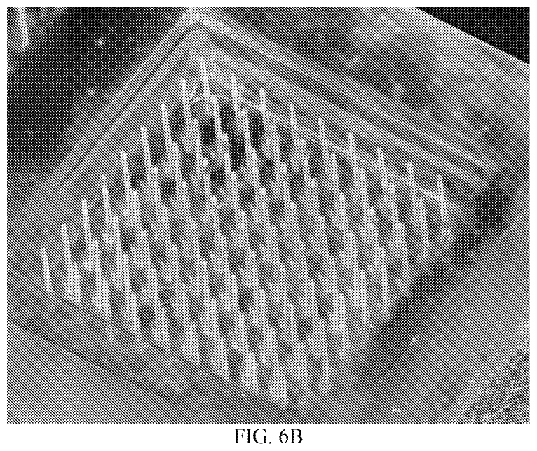

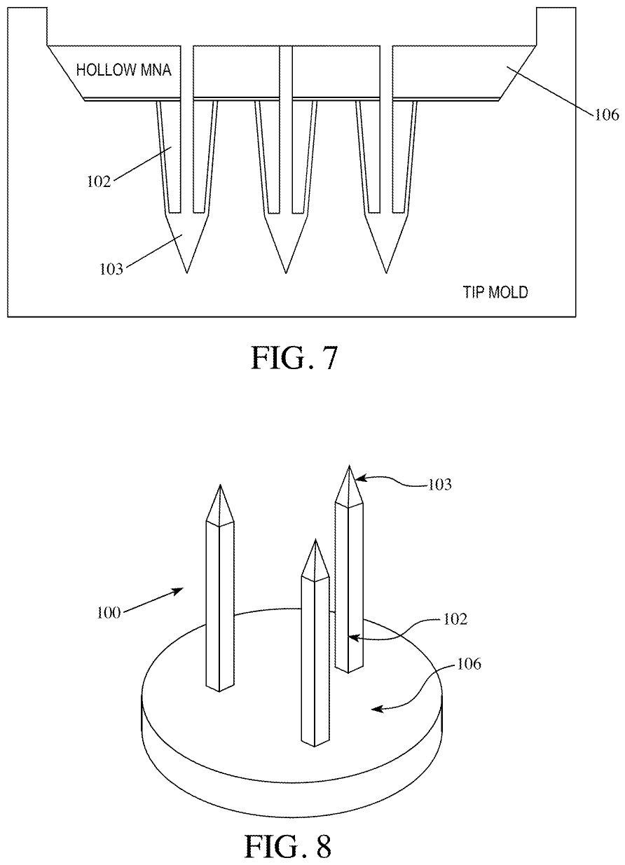

FIG. 5 is a flowchart showing a process for fabricating the microneedle array. At step 201, master molds are created for the microneedle array 100. In one example embodiment, a tip master mold and a separate body master mold are created in a micromilling process. Similarly, the master molds can be created using 3D printing, photolithography, or any other micro-scale fabrication method. An image of a 3D printed body master mold is shown in FIGS. 6A-6B. At step 202, production molds are created from the master molds. Typically, the production molds are created with an elastomeric material. However, a person having skill in the art will recognize that various material can be used for the production molds. Of note, the master molds replicate the final structure of the array 100 and the production molds are negative molds. At step 203, a dissolvable material is deposited into a portion of the tip production mold. The material can be deposited via a gravity-fill, spin-casting, or vacuum-assist. At step 204, the body 102 of the microneedle 101 is fabricated in the body production mold. The body 102 is molded with UV-cured or thermally-cured resin, a thermo-plastic, or another type of material, wherein the mold is filled with the liquid-phase polymer, cured, then demolded in solid form. At step 205, the solid body 102 portion of the microneedle 101 is inserted into the tip production mold. The distal end of the body 102 will contact the tip material and adhere as the material solidifies. To improve the adhesion between the body 102 and tip 103, additional tip material can be added to the cavity of the body 102 in an option fabrication step. Adhesion can also be improved by shaping or roughening the distal end of the body 102. In addition, the assembled system may be placed in centrifuge or vacuum to aid the adhesion and creation of the tip shapes. After adhesion, the microneedle 101 is demolded from the tip mold. Demolding can be facilitated by passivating the surface of the molds with a low surface energy cleaning and coating, such as such as plasma cleaning and using TFOCTS/PFOCTS (tridecafluoro-1,1,2,2-tetrahydrooctyl-1-trichlorosilane) or other silanes. To aid alignment of the body 120 with the recesses of the tip mold, the base 106 of the array 100 is chamfered (˜15 degrees) to engage a corresponding chamfer on the mold, as shown in FIG. 7. The taper of the body 120 also aids alignment and guides the microneedle 101 into the mold opening. Further shown in FIG. 7, the body 102 is tapered to facilitate insertion into the tip mold, which has an opening slightly larger than the body 102. For example, in one embodiment, the opening of the mold is 10 μm larger than the body 102.

By way of further detail, an example fabrication process is described as follows. At step 201, two master molds are mechanically micro-machined out of a hard polymer (e.g., Polymethyl methacrylate (PMMA))—one for the hollow body 102 and another for the dissolvable tips 103. The master mold material may be any easily machinable material such as metal (i.e. aluminum) or plastic (i.e. PMMA, curable resins, etc.) allowing a wide range of geometries. Micromachining methods may include mechanical micromilling, lithography, or micro electrode-discharge machining to make the molds from a variety of materials including plastics, ceramics or metals (including stainless steel, aluminum, copper, iron, tungsten, and their alloys). In another embodiment, the master molds are created using 3D printing, including SLA, Nanoscribe, and similar approaches. Micromolding is then used to create elastomer production molds from the master molds. In this example, production molds are created from Polydimethylsiloxane (PDMS), but other elastomers or any material with sufficient low surface energy can be used to allow easy demolding.

At step 202, the dissolvable tips 103 are fabricated by spin casting in a centrifuge. During this step, a biodissolvable/biodegradable polymer in a hydrogel form is loaded into the elastomer production mold for creating the tips 103. At step 203, the body 102 is created through depositing a biocompatible UV-curable resin in the elastomer production mold. This step can also be done by using thermoplastics or other type of thermoset plastics.

After spin casting the tips 103 inside a centrifuge for a short time and removing the excess hydrogel, the hollow body 102 made of a cured resin is inserted on top of the dried tips 103 into the same elastomer mold. An additional amount of polymer can be inserted from the top and spin dried again to produce the final microneedle array 100. The biopolymers used for the tips in this example embodiment are carboxymethyl cellulose (CMC) and polyvinyl alcohol (PVA) hydrogels. The assembled system is then placed in a centrifuge for the required duration for tips to fully dry.

In a second fabrication process, the master mold production could be replicated using microfabrication procedures such as deep reactive ion etching to make silicon, silicon dioxide, silicon carbide, or metalized molds. Also, LIGA (i.e. a ‘lithography, electroplating, and molding’ process) or deep UV processes can be used to make molds and/or electroplated metal molds. The molding step can be skipped all together and the hollow body 102 may be directly fabricated from a silicon die, which can be etched in the microfabrication process to create hollow microneedles 101. Alternatively, the master mold or the array 100 can be created using high precision additive manufacturing, such as by using Nanoscribe or BMF3D systems. The drug reservoir 104 may be fabricated inside the silicon die, or an additional thick film layer can be bonded or attached over the silicon substrate to create the reservoir 104.

In addition to drug delivery, the microneedle array 100 can be used for interstitial, blood, oral, and other mucosal sampling. When used for sample, fluid flows from the distal end of the tip 103 through the body 102 into the reservoir 104. To assist with fluid recovery, an absorbent material (such as paper or an absorbent polymer) is loaded into the reservoir 104. After application to the skin and dissolution of the tips 103, the sample is collected by the absorbent material. Alternatively, a continuous sample collection can be used via the adapter 105 and syringe or similar collection mechanism. Sampling via interstitial fluid (ISF) is promising as for diagnosing disease. The microneedle array 100 is particularly suited for collection of ISF as the dermis is 70% ISF by volume and ISF has 3× the cancer markers of plasma.

FIG. 8 depicts an alternative embodiment of the microneedle array 100 adapted for a use not on the skin, but rather the inner ear. There are a broad range of hearing diseases and conditions that respond to drugs delivered into the inner ear. However, current approaches deliver drugs to the middle ear and rely on diffusion through the round window membrane into the scala tympani. This approach can lead to higher treatment doses, reduced specificity, and ototoxicity. Importantly, neither the time course of delivery and pharmacokinetics nor the delivery dosage can be controlled with this approach.

As shown in FIG. 8, the array 100 includes three microneedles 101 placed on a circular backing. The needles 101 have an obelisk shape and with 100-250 μm width and 0.75-1.5 mm height. This array 100 will include rapidly dissolving tips 103 that will dissolve within 10-30 mins after insertion. These tips 103 will be made from a combination of carboxymethyl cellulose and trehalose. The body 102 of the needles 101, as well as the backing will include a non-dissolvable shell. Inside the needles 101 and the reservoir 104 is poly(lactic-co-glycolic acid) with a mixed drug comprising gentamicin and dexamethasone. Formulations with varying polylactic acid to glycolic acid ratios (e.g., 75:25) can be chosen to enable varying the total dissolution period. This example array 100 can incorporate 4 mg of drug with customizable delivery profile. The resin body 102 and the CMC/Trehalose tip 103 will provide the necessary strength for needles to penetrate through the round window membrane without failure.

In yet another alternative embodiment, the array 100 can be combined with the application of an electric field between an anode and cathode attached to the skin causing a low-level electric current. The iontophoresis augmentation can provide the necessary means for molecules to travel through the thicker dermis into or from the body, thereby increasing the permeability of both the stratum corneum and deeper layers of skin. While the transport improvement through the stratum comeum is mostly due to microneedle piercing, iontophoresis can provide higher transport rates in epidermis and dermis.

The hybrid microneedle arrays 100 bring important advantages for vaccination over traditional intradermal delivery systems, including (1) targeted skin delivery with consistent reproducibility, enabling considerable dose-sparing and lower toxicity; (2) precision delivery of the vaccine to a defined skin microenvironment, increasing sustained bioavailability and facilitating development of a robust adaptive immune response; (3) capability to delivery many vaccine types, including replication-competent and/or live-attenuated vaccines; (4) fabrication and sterilization independent of the vaccine, protecting vaccine potency and streamlining regulatory approval; (5) simple, pain-free application requiring no special training; (6) cost-effective, scalable, and flexible fabrication approaches; and (7) minimizing cold-chain space requirements and eliminating biohazardous sharps waste.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof. In particular, one or more features in any of the embodiments described herein may be combined with one or more features from any other embodiments described herein.

Protection may also be sought for any features disclosed in any one or more published documents referred to and/or incorporated by reference in combination with the present disclosure.

What is claimed is:

1. A microneedle for delivering materials into a patient comprising:
a body having a proximate end and a distal end, wherein a cavity traverses an interior of the body along a longitudinal axis from the proximate end to the distal end;
a molded tip comprising a dissolvable material attached to the distal end of the body; and
a reservoir having a volume capable of holding a bioactive agent, wherein the reservoir is in fluid communication with proximate end of the body.

2. The microneedle of claim 1, wherein a plurality of microneedles are arranged in an array.

3. The microneedle of claim 2, wherein each microneedle of the plurality is arranged in a grid with equal spacing between each microneedle.

4. The microneedle of claim 1, wherein the materials are delivered into a tissue selected from a group consisting of skin tissue, heart tissue, inner ear tissue, cancerous tissue, diseased tissue, and eye tissue.

5. The microneedle of claim 1, where the microneedle is fitted with an adapter to facilitate delivery of liquid or solid bio-cargo from the back side of the adapter using a standard Luer Tip or Luer Lock syringe.

6. The microneedle of claim 1, wherein the body has a round or square cross-sectional shape.

7. The microneedle of claim 1, wherein the tip is pyramid-shaped or cone-shaped.

8. The microneedle of claim 1, wherein the tip has a base diameter larger than a diameter of the body.

9. The microneedle of claim 1, further comprising a layer of poly(lactic-co-glycolic acid) disposed in the distal end of the body.

10. The microneedle of claim 1, wherein the body has a negative angle to create a retaining geometry.

11. The microneedle of claim 1, wherein the distal end of the body is shaped or roughened to facilitate attachment of the tip to the body.

12. The microneedle of claim 1, wherein the cavity of the body is tapered to facilitate demolding during fabrication.

13. The microneedle of claim 1, further comprising a layer of poly(lactic-co-glycolic acid) disposed in the distal end of the body to separate the tip from the material.

14. The microneedle of claim 1, wherein the dissolvable material comprises at least one of polyvinyl alcohol, carboxymethlycellulose, trehalose, glucose, maltose, PVP, and maltodextrin.

15. The microneedle of claim 1, wherein the dissolvable tip has an encapsulated compound of a drug as a secondary phase delivery.

16. The microneedle of claim 1, wherein the dissolvable tip encapsulates an adjuvant or an anti-inflammatory compound.

17. The microneedle of claim 1, wherein the delivered material is selected from the group consisting of an RNA, mRNA, DNA, protein, viral, inactivated, or live-attenuated virus-based vaccine.

18. The microneedle of claim 1, wherein the microneedle is used for delivery into the oral cavity (buccal delivery), sublingually, or other mucosal membranes.

19. A method of fabricating a hybrid microneedle array comprising:
forming a tip mold and a body mold;
filling a portion of the tip mold with a dissolvable material;
using the body mold, molding a body having a cavity traversing the longitudinal axis of the body;
placing the body into the tip mold having the dissolvable material, wherein the body adheres to the dissolvable material; and
demolding the microneedle array comprising the body with an adhered tip.

20. The method of claim 19, further comprising:
adding dissolvable material to the cavity of the body prior to demolding.

21. The method of claim 19, wherein forming a tip mold comprises:
forming a master tip mold; and
forming a production tip mold from the master tip mold.

22. The method of claim 19, wherein forming a body mold comprises:
forming a master body mold; and
forming a production body mold from the master body mold.

23. The method of claim 19, further comprising:
forming an integrated reservoir adjacent to the body.

24. The method of claim 19, where an adapter is co-fabricated with the hybrid microneedle array.

25. The method of claim 21 or 22, wherein the master tip mold or master body mold is fabricated by mechanical micromachining or 3D printing.

26. The method of claim 19, wherein the tip mold and body mold are passivated using a low surface energy cleaning and coating.

27. A product formed by the method of any of claims 19-26.
